(19) United States
(12) Patent Application Publication
(10) Pub. No.: US 2015/0335355 A1
(43) Pub. Date: Nov. 26, 2015

(54) CALLOUS SCRAPER

(71) Applicant: Stephen W. Belch, SR., Creswell, NC (US)

(72) Inventor: Stephen W. Belch, SR., Creswell, NC (US)

(21) Appl. No.: 14/283,605

(22) Filed: May 21, 2014

(51) Int. Cl. A61B 17/54; A45D 29/18

(52) U.S. Cl. CPC A61B 17/54; A45D 29/18; A61B 2017/320008

(57) ABSTRACT

A callous remover includes a spherical shaped device; an abrasive surface over the spherical shaped device; at least two supports along a circumference of the spherical shaped device; openings between the supports; and a means for mounting along the circumference of the spherical shaped device. The means for mounting includes two opposing screws along the circumstance of the spherical shaped device.

### BACKGROUND OF THE INVENTION

1. Field of Invention

The present invention relates to a callous scraper that is mountable in a bathtub or shower for convenient use.

2. Description of Related Art

Frequently individuals develop a callous over their feet due to repeated friction within a shoe or otherwise. Many times callous can develop into a thick layer and be difficult to remove from the surface of a person's foot. Callous normally develops around the heel and the ball of the toe areas of the foot. These areas of the foot normally absorb a significant amount of contact and friction during walking and standing. In order to remove a callous, individuals use various files and scales that normally include an abrasive surface. Further individuals soak their feet in soapy water to help soften the callous and therefore remove it over a period of time. Certain abrasive paper and cloths are available to help remove a callous from an individual's foot. As a consequence, currently there are various methods to address this problem that is frequently suffered by many individuals.

### SUMMARY OF THE INVENTION

The present invention relates to a callous remover comprising: a spherical shaped device; an abrasive surface over the spherical shaped device; at least two supports along a circumference of the spherical shaped device; openings between the supports; and a means for mounting along the circumference of the spherical shaped device. The means for mounting includes two opposing screws along the circumstance of the spherical shaped device.

### BRIEF DESCRIPTION OF DRAWINGS

FIG. 1 depicts a callous scraper in accordance with the present invention.

### DETAILED DESCRIPTION

The present invention relates to a callous scraper in the form of a spherical shaped abrasive surface. The spherical surface includes a scraping area so that an individual can scrape their feet on the scraper to remove a callous from the hard-to-reach areas of their feet. The callous scraper according to present invention is mountable in a bathtub or shower area and includes mounting screws with support structures to support the callous scraper in a stationary position mounted within the shower area.

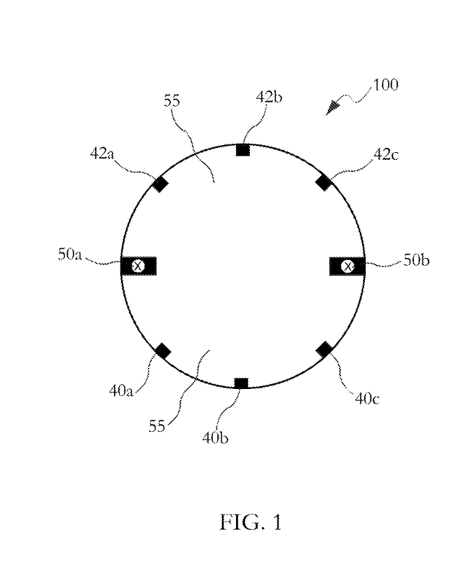

In reference to FIG. 1 a Callous Scraper 100 is depicted. This Callous Scraper 100 includes mounting screws 50a, 50b at opposing sides of the callous scraper 100. The callous scraper 100 has a spherical shape. Along the outside radius of the scraper 100 are equally spaced supports that support the scraper 100 in a stationary position once mounted with the mounting screws 50a, 50b. Top supports 42a, 42b, 42c are shown in FIG. 1 along with the bottom supports 40a, 40b, 40c. These supports are equally spaced along the outer circumference of the Scraper 100. The Scraper 100 further includes an abrasive surface 55. This abrasive surface 55 provides an open abrasive surface so that an individual may place their feet on this surface and enable the removal of the callous on the individual's foot. The spherical shape helps to address various angles and positions of the foot while maneuvering the foot for a callous removal.

The Scraper 100 therefore helps to remove dead skin along with the callous to provide for a smoother skin of an individual's foot. In one particular embodiment, this scraper is placed directly over the drain of the bathtub or shower. Openings are provided between the supports to allow for the movement of water under the Scraper 100 and therefore allow for convenient positioning of the scraper in the bathtub or shower. Once mounted in place with the mounting screws, the scraper is available for use and easy removal of the callous with disposal of the removed callous through the flow of water into the drain below the scraper.

With respect to the bathtub application, the Scraper 100 preferably may be mounted to the side of the tub, above the water line. All bathtubs have a drain built in the upper portion of the tub, to prevent the tub from over flowing. The Scraper 100 may be mounted above the line, of this drain, to prevent any possible leakage around the mounting screws. In one particular embodiment, the Scraper 100 may be mounted with two (2) stainless steel screws, two (2) ¼″ rubber spacers and two (2) stainless steel butter fly nuts. The desired mounting location may be marked, next two (2) holes are drilled, large enough to allow the insertion of the butter fly nuts. A user may then place the screws through the Scraper 100, put the ¼″ rubber spacers on the screws and insert the butter fly nuts on the screws. Next the user pushes the screws, with nuts in place, through the drilled holes. After the screws and nuts are within the holes, the user may place some silicone around the holes before completely tightening the two screws. The silicone seals the holes to prevent any water from leaking through the tub.

Preferably, the Scraper 100 is mounted on each side of the tub, so that the user may clean the calluses' from each foot, while reclining in a comfortable position within the bathtub. The present invention enables the user to clean the underside of their feet without having to bend down and use a scraper, a file or other handheld device. The removal of the callous can be accomplished in the standing position while in the shower or bathtub. This invention further helps to eliminate the need for any additional cleanup of callous or dead skin that is removed from the individual's foot. The instant invention has been shown and described in what it considers to be the most practical and preferred embodiments. It is recognized, however, that departures may be made there from within the scope of the invention and that obvious modifications will occur to a person skilled in the art.

What is claimed is:

1. A callous remover comprising:
a. a spherical shaped device;
b. an abrasive surface over the spherical shaped device;
c. at least two supports along a circumference of the spherical shaped device;
d. openings between the supports; and
e. a means for mounting along the circumference of the spherical shaped device.

2. The callous remover according to claim 1, where the mounting means includes two opposing screws along the circumstance of the spherical shaped device.

3. The callous remover according to claim 1, where the at least two supports include three top supports and three bottom supports.

4. The callous remover according to claim 3, where evenly spaced openings are provided between the supports.

5. The callous remover according to claim 1, where the callous remover is installed directly over a drain during use.
